(19) United States
(12) Patent Application Publication
(10) Pub. No.: US 2026/0013716 A1
(43) Pub. Date: Jan. 15, 2026

(54) Vaginal Speculum

(71) Applicant: The University of Toledo, Toledo, OH (US)

(72) Inventors: Shelby Marshall, Toledo, OH (US); Priyanka Pulvender, Toledo, OH (US); Alekhya Varada, Toledo, OH (US); Nicole Valentina Campos Yanez, Toledo, OH (US); Lana Asma Alaraje, Toledo, OH (US)

(73) Assignee: The University of Toledo, Toledo, OH (US)

(21) Appl. No.: 19/265,002

(22) Filed: Jul. 10, 2025

Related U.S. Application Data
(60) Provisional application No. 63/669,377, filed on Jul. 10, 2024.

(51) Int. Cl. A61B 1/32; A61B 1/303

(52) U.S. Cl. CPC A61B 1/32; A61B 1/303

(57) ABSTRACT

A speculum having two blades openable in a horizontal manner with respect to a body cavity, and methods for using the speculum, are described. The speculum includes a mount extending from a top surface of a first handle. The mount comprises a head portion and a neck portion extending between the top surface of the first handle and the head portion. In certain embodiments, the head portion is circular and defines an opening configured to receive a light. The opening may be substantially centered within a tubular structure formed by edges of the first blade and the second blade when the speculum is in a closed state. The light may be disposed within the mount and comprise a first portion and a second portion. The light first portion may be positioned on a mount distal side and the light second portion may be positioned on a mount proximal side.

### RELATED APPLICATIONS

This application claims priority to U.S. Provisional Application No. 63/669,377 filed under 35 U.S.C. § 111(b) on Jul. 10, 2024, the disclosure of which is incorporated herein by reference in its entirety.

### STATEMENT REGARDING FEDERALLY SPONSORED RESEARCH

This invention was made with no government support. The government has no rights in this invention.

### BACKGROUND

Vaginal speculums are medical devices commonly used during gynecological examinations to visualize and access the cervix and vaginal canal. Traditionally, speculums have been constructed from metal, featuring two hinged blades that can be opened and closed by a handle mechanism. However, vaginal speculums can be uncomfortable for patients. There is a need for new and improved vaginal speculums.

### SUMMARY

Provided herein is a speculum comprising a first member having a first blade and a first handle, the first blade extending from a first blade proximal end to a first blade distal end; a second member having a second blade and a second handle, the second blade extending from a second blade proximal end to a second blade distal end; and a fastener configured to be disposed through the first handle and the second handle so as to secure the first member to the second member in a manner that allows the first blade and the second blade to open and close, the first handle and the second handle being movable so as to transition the speculum between an open state in which the first blade and the second blade are a first distance apart, and a closed state in which the first blade and the second blade are a second distance apart, wherein the first distance is greater than the second distance.

In certain embodiments, the first blade is tapered from a first blade proximal end to a first blade distal end, and the second blade is tapered from a second blade proximal end to a second blade distal end. In particular embodiments, the tapering of the first blade is uniform, and the tapering of the second blade is uniform.

In certain embodiments, the first blade is curved, and the second blade is curved.

In certain embodiments, the first handle has a first handle distal end and the first blade a first blade inferior edge, and the first blade inferior edge is attached to a first corner of the first handle distal end; and the second handle has a second handle distal end and the second blade has a second blade inferior edge, and the second blade inferior edge is attached to a second corner of the second handle distal end.

In certain embodiments, the first blade has a first convex surface, the second blade has a second convex surface, and either the first convex surface or the second convex surface includes at least one marking configured to serve as a measurement indicator.

In certain embodiments, the first member further comprises a mount extending from a top surface of the first handle. In particular embodiments, the mount comprises a neck portion and a head portion, the neck portion extending between the top surface of the first handle and the head portion. In particular embodiments, the head portion is circular and defines an opening configured to receive a light. In particular embodiments, the opening is substantially centered within a tubular structure formed by edges of the first blade and the second blade when the speculum is in the closed state. In particular embodiments, the speculum further comprises a light system disposed within the mount. In particular embodiments, the light system comprises a light first portion and a light second portion, the light first portion being positioned on a mount distal side and the light second portion being positioned on a mount proximal side, wherein the light first portion houses a light and the light second portion houses a power source in electrical communication with the light.

In certain embodiments, the fastener is configured to adjust tension between the first member and the second member.

In certain embodiments, the first blade has a first length from the first blade proximal end to the first blade distal end, and the second blade has a second length from the second blade proximal end to the second blade distal end, the second length being longer than the first length.

In certain embodiments, the first blade has a first convex surface, the second blade has a second convex surface, and the first convex surface and the second convex surface are smooth.

In certain embodiments, the speculum further comprises a sleeve covering at least a portion of the first blade and/or the second blade. In particular embodiments, the sleeve is silicone or latex.

In certain embodiments, the fastener comprises a nut and bolt.

Further provided is a method of examining a body cavity, the method comprising actuating first and second handles of a speculum so as to cause first and second blades of the speculum to move closer together, thereby causing the speculum to be in a closed state; inserting a distal end of the first and second blades into the body cavity; moving the first and second blades a desired amount into the body cavity; allowing the first and second handles to move farther apart so as to cause the first and second blades to move farther apart in a horizontal direction relative to the body cavity, thereby causing the speculum to be in an open state; and examining the body cavity.

In certain embodiments, the method further comprises illuminating the body cavity with a light mounted at the distal end of the speculum.

In certain embodiments, the method further comprises actuating the first and second handles so as to cause the first and second blades to move closer together, thereby causing the speculum to be in the closed state, and removing the first and second blades from the body cavity.

Further provided is a speculum comprising first and second blades extending in a direction orthogonal to first and second handles, the handles being pivotable around aligned bores receiving a fastener, the fastener being configured to lock or unlock the first and second handles in position relative to one another; wherein the first and second blades are moved closer to one another as the first and second handles are pivoted to a greater extent; and wherein the first and second blades are curved so as to define a tubular structure when the speculum is in a closed state.

In certain embodiments, the speculum further comprises a light mounted to the first handle at a distal end of the tubular structure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Perspective view of a non-limiting example embodiment of a vaginal speculum.

FIG. 2: Perspective view of a non-limiting example embodiment of a first member of the vaginal speculum.

FIG. 3: Perspective view of a non-limiting example embodiment of a second member of the vaginal speculum having a light system exploded from the second member.

FIG. 4: Side view of the non-limiting example embodiment of the vaginal speculum.

FIG. 5: Side view of the non-limiting example embodiment of the vaginal speculum.

FIG. 6: Perspective view of the non-limiting example embodiment of the vaginal speculum.

FIG. 7: Perspective view of the non-limiting example embodiment of the vaginal speculum.

FIG. 8: Perspective view of the non-limiting example embodiment of the vaginal speculum.

FIG. 9: Perspective view of the non-limiting example embodiment of the vaginal speculum.

FIG. 10: Side view of the non-limiting example embodiment of the vaginal speculum in a closed position.

FIG. 11: Side view of the non-limiting example embodiment of the vaginal speculum in an open position.

FIG. 12: Perspective view of a non-limiting example embodiment of a sleeve.

FIG. 13: Perspective view of the non-limiting example embodiment of the sleeve.

FIG. 14: Perspective view of a non-limiting example embodiment of a first sleeve member and a second sleeve member.

FIG. 15: Perspective view of the non-limiting example embodiment of the first sleeve member and the second sleeve member.

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

Provided herein is a speculum with features for improved comfort of patients and easier usability for practitioners. The speculum is useful for viewing a vaginal canal or other body cavity of the patient. Although the device is particularly useful for viewing a vaginal canal, and may be referred to herein as a vaginal speculum, the speculum can be modified and used in any desired type of procedure where a body cavity is opened or viewed.

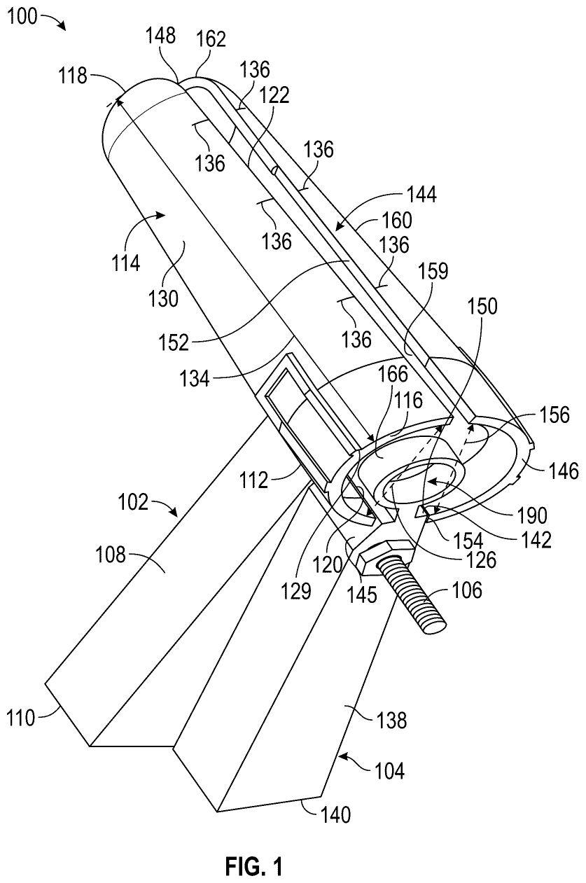

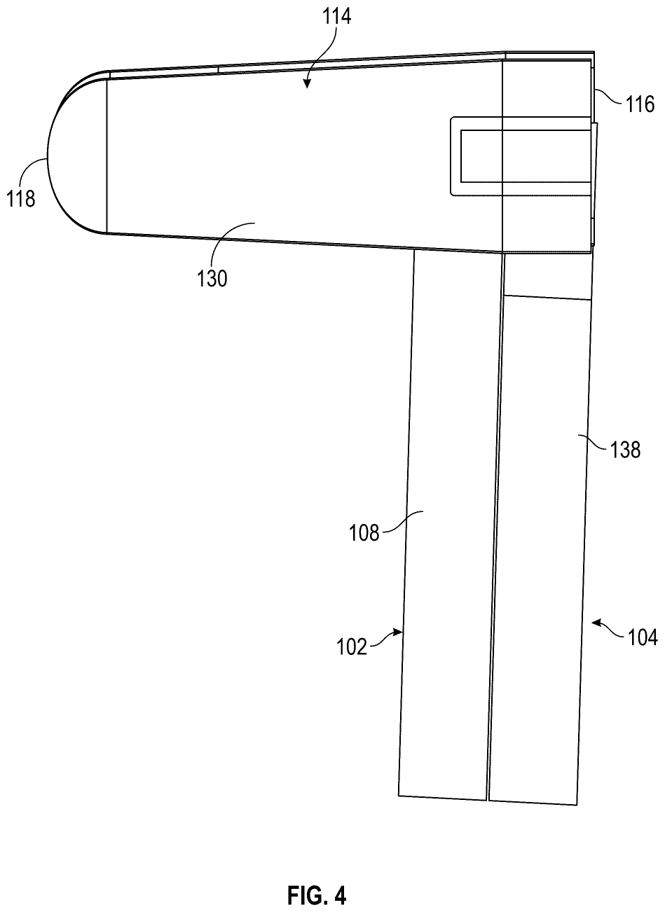

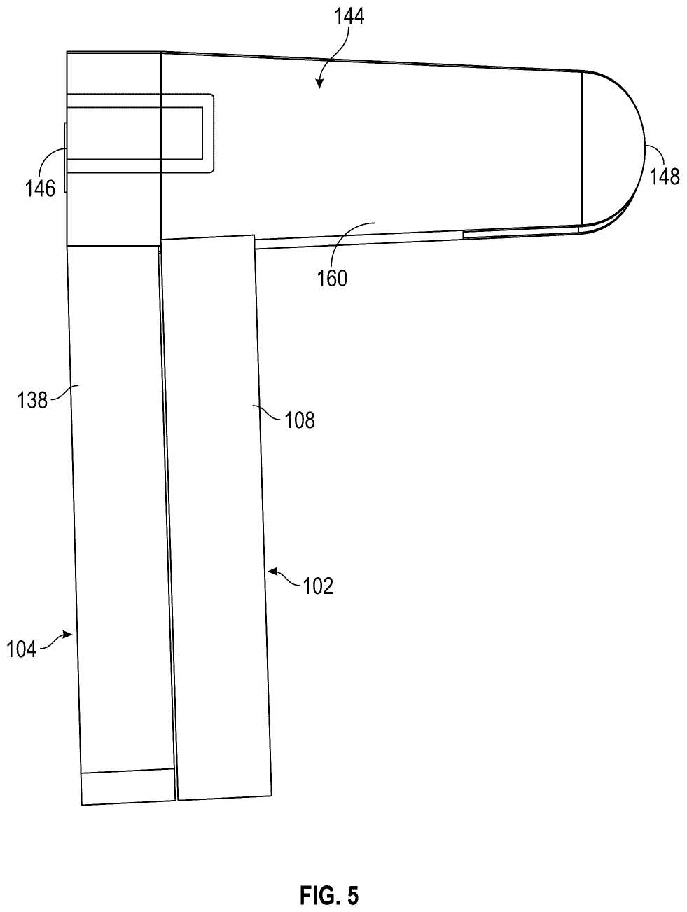

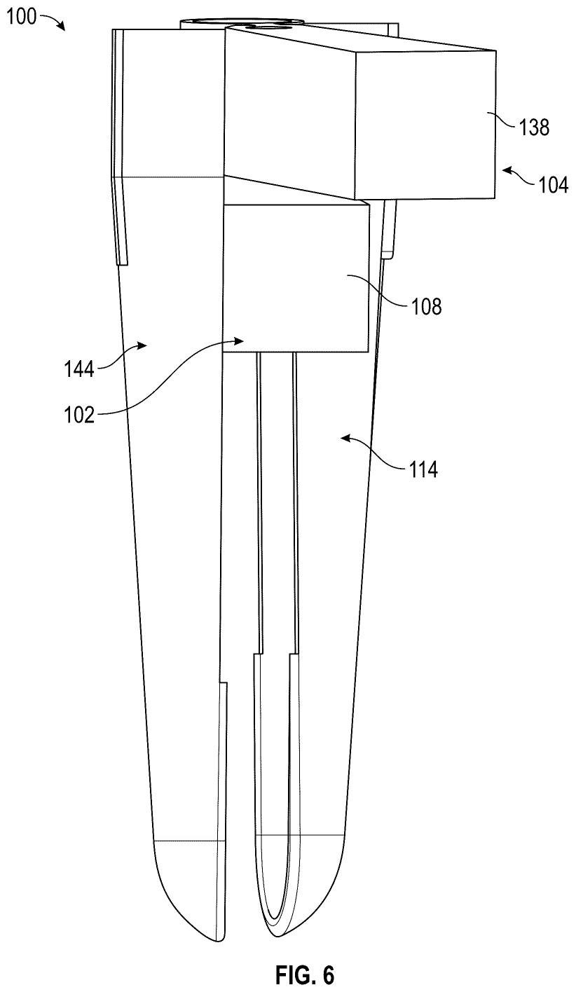

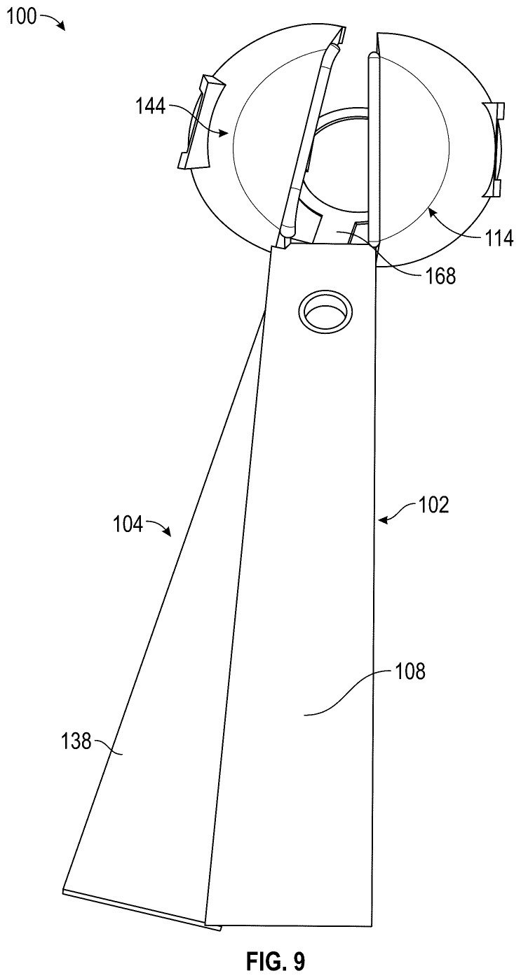

Referring now to FIGS. 1 and 4-11, an embodiment of a speculum 100 is depicted. The speculum 100 has a first member 102 and a second member 104. The first member 102 and the second member 104 are removably connected by a fastener 106. The fastener 106 may be a bolt or screw, though other fasteners are possible and encompassed within the scope of the present disclosure.

Referring now to FIGS. 1-2 and 4-11, the first member 102 has a first handle 108 extending between a first handle bottom 110 and a first handle top 112. The second handle 108 may have a rectangular cross section, although this is not strictly necessary. The second handle 108 may also further include gripping elements to aid a practitioner's grip of the second handle 108.

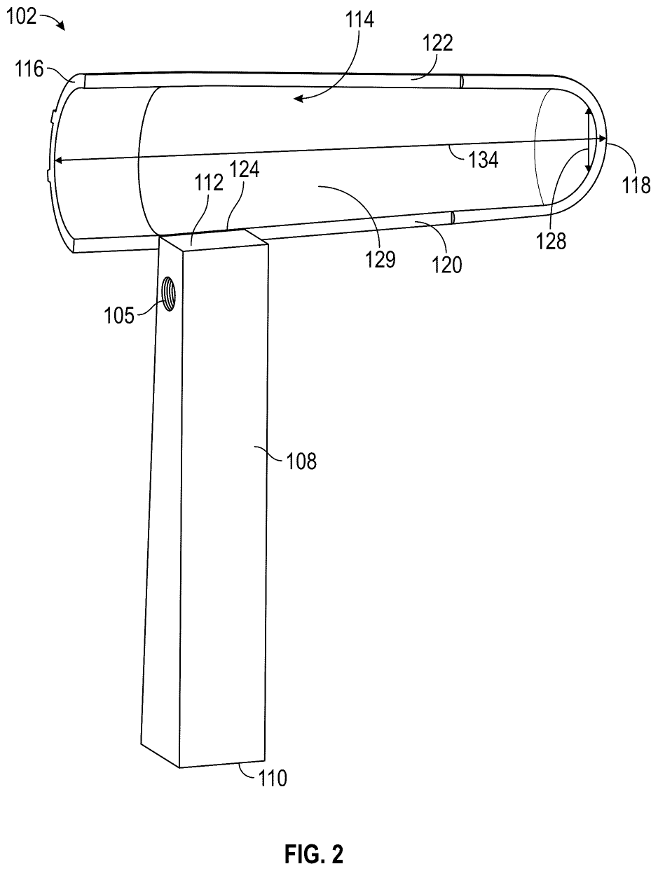

Referring still to FIG. 2, the first member 102 includes a first blade 114 having a first blade proximal end 116, a first blade distal end 118, a first blade inferior edge 120, and a first blade superior edge 122. The first blade 114 may be curved so as to form an arcuate or semi-circular cross section. A portion of the first blade inferior edge 120 may join a first corner 124 of the first handle top 112, although the first blade 114 can be joined to the first handle top 112 at any desired position. Joining the first blade 114 at the first corner 124 is advantageous at least because it allows for smooth operation of the speculum 100. The first member 102 can be two separate pieces conjoined, or alternatively the first member 102 can be one uniform piece. Thus, the term “joined” is used to refer to two components meeting in a continuous manner or being affirmative connected or attached. In the illustrated embodiment, the first handle 108 is rectangular. However, the first handle 108 can be any shape including, but not limited to, cylindrical, ovular, or a custom shape having a grip.

The first blade 114 may be tapered in thickness, diameter, or both from the first blade proximal end 116 to the first blade distal end 118 such that the first blade proximal end 116 has a first diameter 126 which is greater than the second diameter 128 of the first blade distal end 118 and/or the first blade proximal end 116 has a thickness which is greater than a thickness at the first blade distal end 118. The tapering of the first blade 114 may be uniform or may be non-uniform. In other words, the tapering may be non-monotonic. The tapering of the first blade 114 is configured to allow a single speculum to be used for any vaginal canal size. The first blade 114 may also include a curvature, for example by being semi-circular and having a first concave surface 129 that extends from the first blade proximal end 116 to the first blade distal end 118. The first concave surface 129 is configured to face a second concave surface 159 of the second blade 144. Because the first concave surface 129 and the second concave surface 159 may be aligned and match in size and concavity, the first blade 114 and the second blade 144 may form a tubular structure when the speculum 100 is in a closed state. The first blade 114 also has a first convex surface 130 that is configured to mimic the anatomy of a vaginal canal. The first convex surface 130 is the outer surface of the first blade 114 and has a smooth surface 131 that has a low coefficient of friction to allow the speculum 100 to slide within a vaginal canal while reducing the discomfort that an individual experiences.

Referring to FIGS. 1 and 4-11, the first blade distal end 118 may include a first rounded end 132. The first rounded end 132 helps prevent uncomfortable contacts with skin or organs. Further, the first blade 114 has a first length 134 that extends from the first blade proximal end 116 to the first blade distal end 118. Also, the first blade proximal end 116 is relatively planar. However, the first blade proximal end 116 need not be planar. The first convex surface 130 may have a singular marking or a plurality of markings 136 to serve as an indication of the diameter of the first blade 114 at each marking. The markings 136 thereby aid the practitioner in avoiding discomfort experienced by the patient.

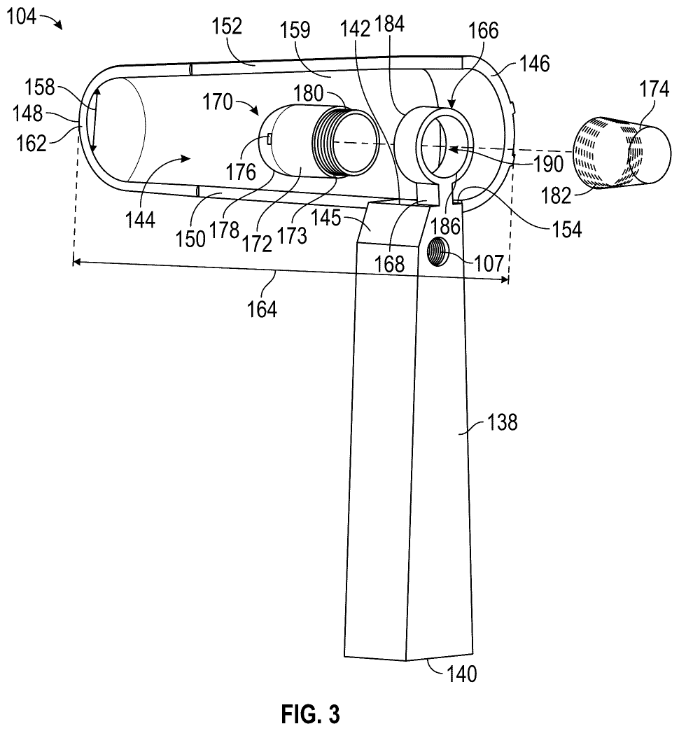

Referring now to FIG. 3, the second member 104 has a second handle 138 extending between a second handle bottom 140 and a second handle top 142. The second handle 138 may have a rectangular cross section, although this is not strictly necessary. The second handle 138 may also further include gripping elements to aid a practitioner's grip of the second handle 138.

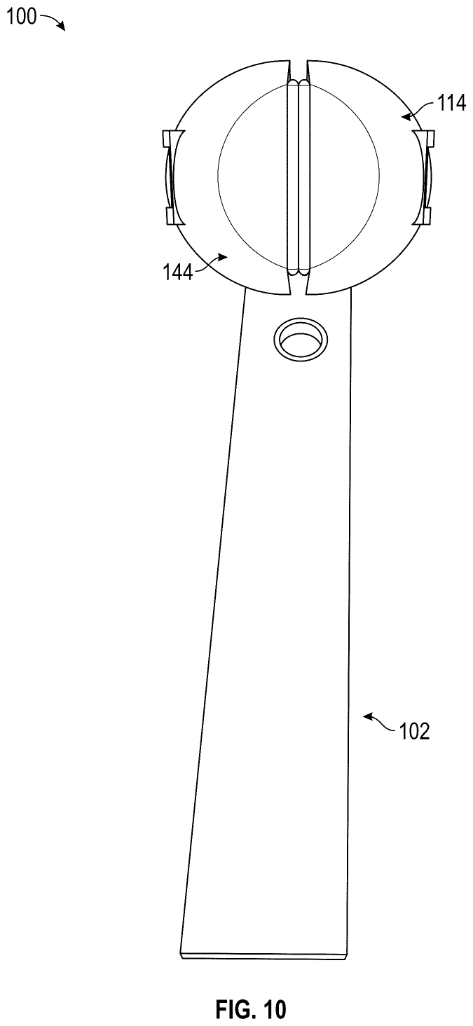

Referring still to FIG. 3, the second member 104 includes a second blade 144 having a second blade proximal end 146, a second blade distal end 148, a second blade inferior edge 150, and a second blade superior edge 152. The second blade 144 may be curved so as to form an arcuate or semi-circular cross section. The curved second blade 144 may come together with the curved first blade 114 so as to form a tubular structure where the first blade inferior edge 120 meets the second blade inferior edge 150 and the first blade superior edge 122 meets the second blade superior edge 152, as seen in FIG. 10. The tubular structure defines a bore into which the mount 166 extends.

Referring still to FIG. 3, a portion of the second blade inferior edge 150 may join a second corner 154 of the second handle distal end 142, although the second blade first edge 150 can be joined to the second handle distal end 142 at any desired position. Joining the second blade 144 at the second corner 154 is advantageous at least because it allows for smooth operation of the speculum 100. The second member 104 can be two separate pieces conjoined, or alternatively the second member 104 can be one uniform piece. Thus, the term “joined” is used to refer to two components meeting in a continuous manner or being affirmative connected or attached. In the illustrated embodiment, the second handle 138 is rectangular. However, the second handle 108 can be any shape including, but not limited to, cylindrical, ovular, or a custom shape having a grip.

Referring still to FIG. 3, the second blade 144 may be tapered in thickness, diameter, or both from the second blade proximal end 146 to the second blade distal end 148 such that the second blade proximal end 146 has a third diameter 156 which is greater than a fourth diameter 158 at the second blade distal end 148 and/or the second blade proximal end 146 has a thickness which is greater than a thickness at the second blade distal end 148. The tapering may be uniform or non-uniform. In other words, the tapering may be non-monotonic. The tapering of the second blade 144 is configured to allow a single speculum to be used for all vaginal canal sizes.

Referring still to FIG. 3, the second blade 144 is semi-circular having a second concave surface 159 that extends from the second blade proximal end 146 to the second blade distal end 148. The second concave surface 159 is configured to face the first concave surface 129. The second blade 144 also has a second convex surface 160 that is configured to mimic the anatomy of a vaginal canal. The second convex surface 160 is the outer surface of the second blade 144 and has a smooth surface 147 that has a low coefficient of friction to allow the speculum 100 to slide within a vaginal canal, reducing the discomfort that a patient experiences during use of the speculum 100. The speculum 100 further has a slanted edge 145 on the second handle top 142 configured to reduce the amount of interference that the second handle top 142 has with the first blade 114.

Referring to FIGS. 1 and 3, the second blade distal end 148 may include a second rounded end 162. The second blade 144 has a second length 164 that extends from the second blade proximal end 146 to the second blade distal end 148. The second length 164 can be relatively longer than the first length 134; however, the second length 164 and the first length 134 can be approximately the same. Also, the second blade proximal end 146 is relatively planar; however, the second blade proximal end 146 need not be planar. The second convex surface 160 may have the singular marking or the plurality of markings 136 as a guide for the practitioner, to serve as an indication of how much of the speculum 100 has been inserted into a vaginal canal.

As seen in FIGS. 2-3, each of the first member 102 and the second member 104 may be an integrally formed piece where the respective blade 114, 144 extends in a direction substantially orthogonal to the respective handle 108, 138. As seen in FIG. 2, the first member 102 includes a bore 105 configured to receive the fastener 106. As seen in FIG. 3, the second member 104 includes a bore 107 configured to receive the fastener 106. The bores 105, 107 are substantially aligned in terms of their respective height along the respective handle 108, 138, so as to cause the blades 114, 144 to be disposed in an aligned manner. The bores 105, 107 form a pivot point around which the first member 102 and the second member 104 may rotate so as to open and close the speculum 100.

Referring now to FIGS. 1-11, the first member 102 and the second member 104 are connected by a fastener 106. In the illustrated embodiment, the fastener 106 is a bolt and a nut. However, the fastener 106 can be any desired fastener 106 that connects the first member 102 and the second member 104 while allowing for the first member 102 and the second member 104 to move relative to one another so as to open and close the blades 114, 144. The fastener 106 not only connects the first member 102 and the second member 104, but the fastener 106 also adjusts the tension between the first member 102 and the second member 104. The tension between the first member 102 and the second member 104 can be increased by tightening the fastener 106, for example when the speculum 100 is inserted in a vaginal canal, so as to retain the speculum 100 in an open state. The fastener 106 can also be tightened when the speculum 100 is in a closed state. The fastener 106 thus allows the speculum 100 to be locked in an open or closed position, preventing the speculum 100 from opening and closing due to the first member 102 and the second member 104 articulating. Also, the first blade 114 and the second blade 144 are configured to open horizontally relative to the handles 108, 138. When the first handle 108 and the second handle 138 are together or near each other, the speculum 100 is in a closed state, and when the first handle 108 and the second handle 138 are separated, the speculum 100 is in an open state.

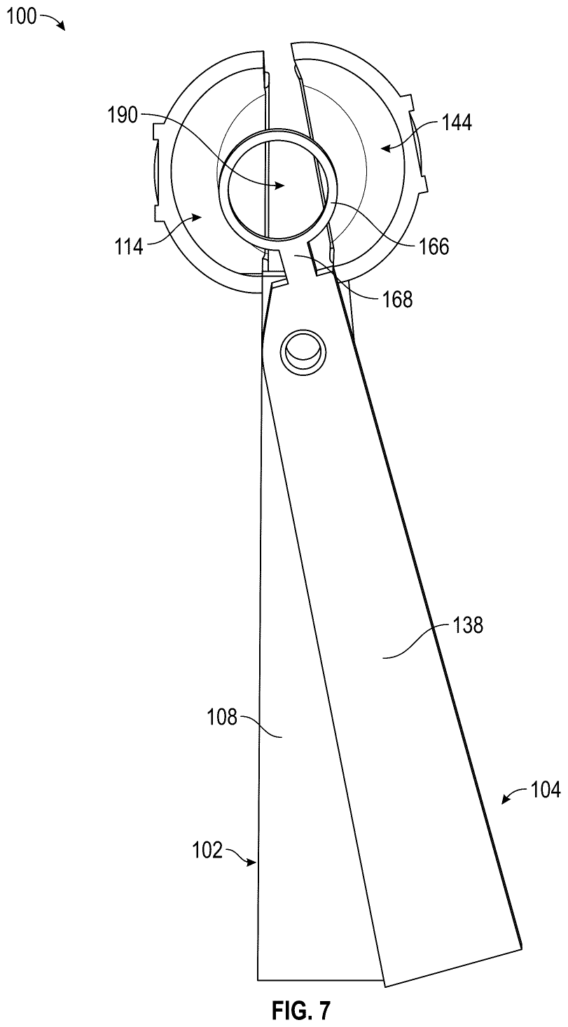

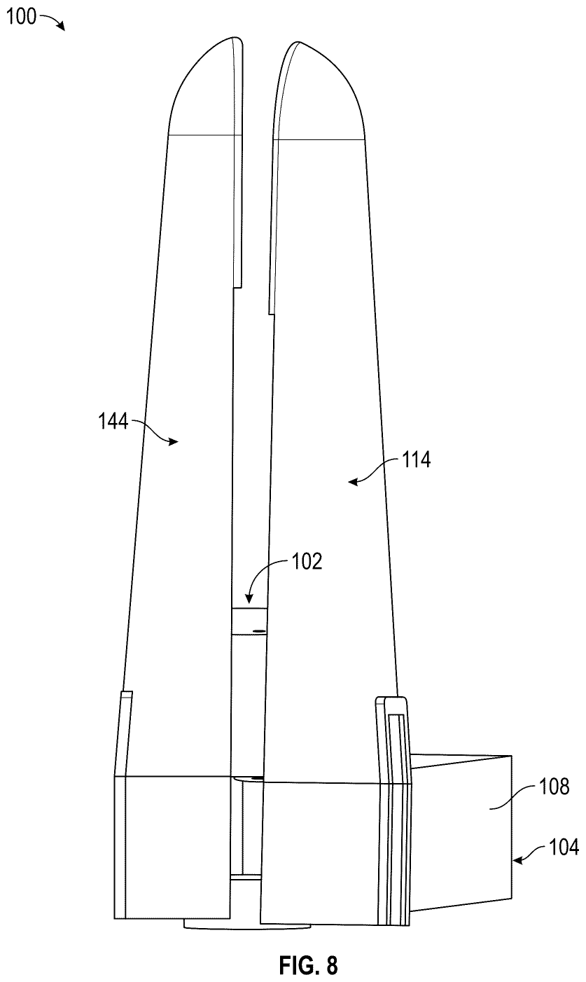

Referring now to FIGS. 7-8, a mount 166 is included on the first member 102, disposed on the first handle 108 at the first handle top 112. The mount 166 includes a neck portion 168 and a head portion 188 defining an opening 190. The neck portion 168 of the mount 166 extends from the second handle top 142 into an area between the edges 120, 122 or the edges 150, 152 at the proximal ends 116, 146 so as to position the head portion 188 to be roughly centered within the tubular structure formed by the first and second blades 114, 144 when the speculum 100 is in a closed state. When the head portion 180 is circular, the head portion 188 may be concentric or nearly concentric with the tubular structure formed by the first and second blades 114, 144 when the speculum is in a closed state. However, as the speculum 100 is moved between an open state and a closed state, the location of the head portion 188 of the mount 166 moves along an arc caused by rotation of the first handle 108 relative to the second handle 138 at the point of the fastener 106.

As best seen in FIG. 8, the head portion 188 of the mount 166 extends from the proximal ends 116, 146 of the blades some distance into the tubular structure in the direction toward the distal ends 118, 148 of the blades 144, 144. In the illustrated embodiment, the head portion 188 is circular; however, the head portion 188 can be any desired shape. Regardless of the shape of the head portion 188, the mount 166 is configured to receive and hold a light configured to illuminate the tubular structure of the speculum 100 in the direction of toward the distal ends 118, 148.

Referring now to FIG. 3, a light system 170 can be inserted into the opening 190 and held in place with a friction fit or optional fasteners. The light system 170 may have a light first portion 172 and a light second portion 174. The light first portion 172 includes a light 176, a light covering 178, and external threads 180. The light 176 can be any kind of light 176 including, but not limited to, a LED or a halogen bulb. The light 176, when disposed in the mount 166, is directed toward the first blade distal end 118 and the second blade distal end 148. The light covering 178 is configured to cover the light 176 in a way that allows light to pass through the light covering 178 and down the first length 134 of the first concave surface 129 and the second concave surface 159. The light first portion 172 has the external threads 180 that are configured to mate with internal threads 182 of the light second portion 174. The external threads 180 and the internal threads 182 may be reversed so that the external threads 180 are on the light second portion 174 and the internal threads 182 are on the light first portion 172. The light second portion 174 is adapted to receive and hold a power source such as a battery or multiple batteries. The light 176 may include conductive feet or wires which extend to, and electrically contact, the power source so as to have power for illumination. The light second portion 174 may further include a switch that can be used to toggle the light 176 on and off. The switch may be a one-way switch or a multiple way switch. If the switch is a multiple way switch, the light 176 intensity can be changed depending on which position the switch is toggled to. The light first portion 172 partially extends through the mount 166 from a mount distal side 184 and the light second portion 174 partially extends through the mount 166 from a mount proximal side 186. The light first portion 172 and/or the light second portion 174 may also have a retaining lip 173 that prevents the light system 170 from detaching from the mount 166.

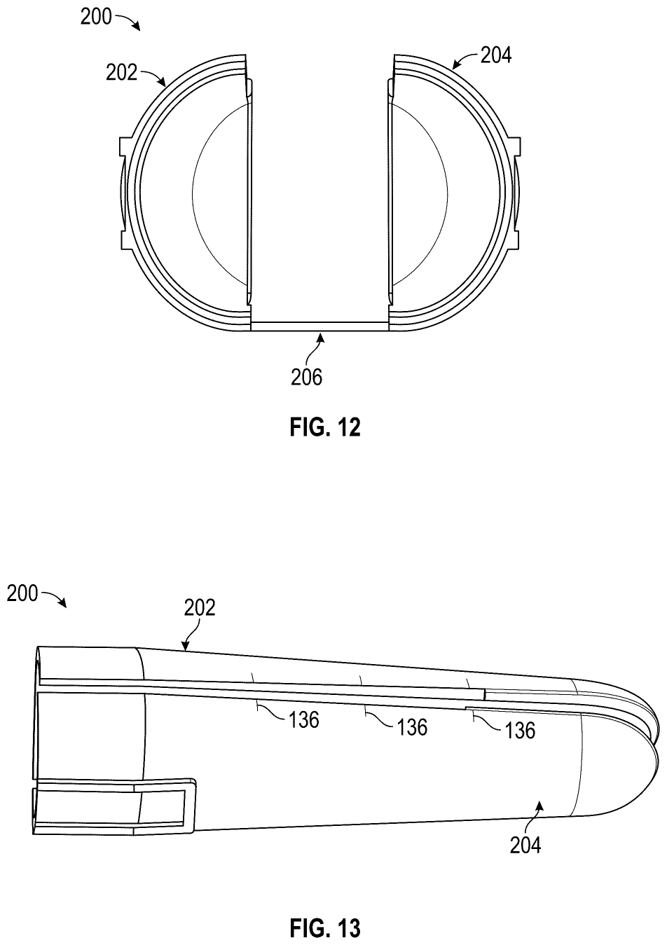

Referring now to FIGS. 12-13, the speculum 100 can further include a removable sleeve 200. The sleeve 200 has a first sleeve member 202, a second sleeve member 204, and a bridge portion 206 that is between the first sleeve member 202 and the second sleeve member 204. The sleeve 200 is configured to be disposed over the first blade 114 and the second blade 144, or over at least a portion of the first blade 114 and at least a portion of the second blade 144. The bridge portion 206 can be made of a different material than the first sleeve member 202 and the second sleeve member 204. The material of the bridge portion 206 can be thinner than the first sleeve member 202 and the second sleeve member 204, allowing the bridge portion 206 to collapse when the speculum 100 is in a closed state and stretch when the speculum 100 is in an open state. Alternatively, the bridge portion 206 can be made from a different material than the first sleeve member 202 and the second sleeve member 204 to allow the bridge portion 206 to collapse when in a closed state and stretch when in an open state. The first sleeve member 202 and the second sleeve member 204 can be made out of any material suitable for insertion into the human body including, but not limited to, silicone or other polymers, or latex. The sleeve 200 can enhance patient comfort, reducing friction between the speculum 100 and tissues (such as in a vaginal canal), and can provide for temperature control of the speculum 100. Moreover, the sleeve 200 is removable and may serve to protect patients from infection. The sleeve 200 can also have a singular marking or a plurality of markings 136 to serve as an indication of the length or diameter of the first sleeve member 202 and second sleeve member 204 at each marking. The sleeve 200 may optionally be lubricated for easier insertion into a body cavity such as a vaginal canal.

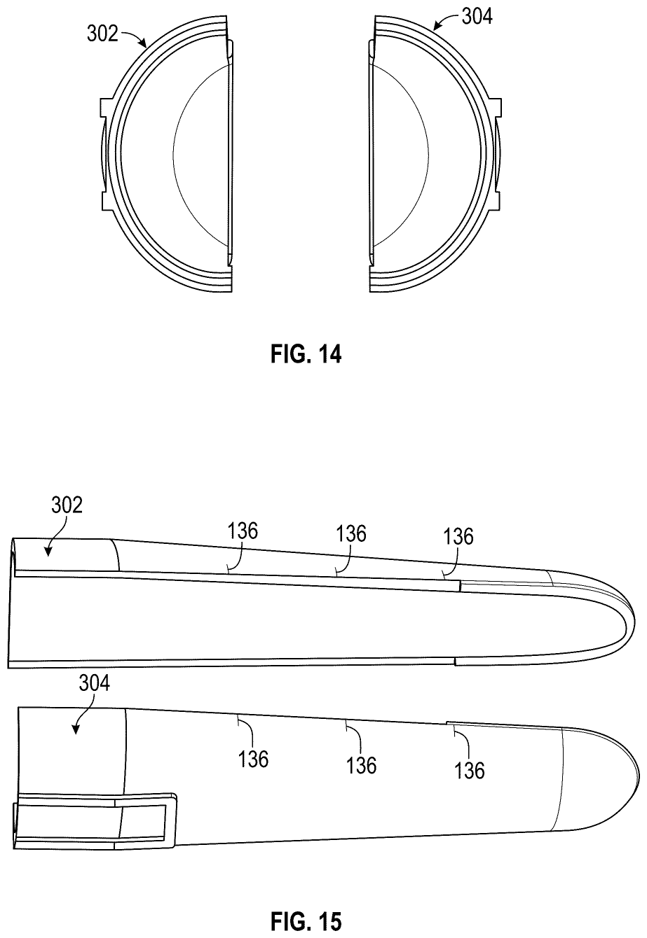

Referring now to FIGS. 14-15, the speculum 100 may include a first sleeve member 302 and a second sleeve member 304 which are configured to be disposed over the first blade 114 and the second blade 144, respectively, or portions thereof, but without a bridge portion connecting the first sleeve member 302 and the second sleeve member 304. The first sleeve member 302 and second sleeve member 304 are different than the first sleeve member 202 and second sleeve member 204 because the first sleeve member 302 and the second sleeve member 304 do not have a bridge portion 206 between the sleeve members 302, 304. The first sleeve member 302 and second sleeve member 304 can be made out of any material suitable for insertion into the human body including, but not limited to, silicone or other polymers, or latex. The first sleeve member 302 and the second sleeve member 304 can be beneficial for a number of reasons including, but not limited to, comfortability, reduced friction between body tissue and the speculum 100, and for temperature control of the speculum 100. The first sleeve member 302 and the second sleeve member 304 can also have a singular marking or a plurality of markings 136 on an exterior surface of the first sleeve member 302 and/or second sleeve member 304 to serve as an indication of the length or diameter of the first sleeve member 302 and second sleeve member 304 at each marking. The first sleeve member 302 and the second sleeve member 304 may include internal gripping features or otherwise be configured to easily slide into a body cavity such as a vaginal canal without inadvertently sliding off of the first blade 114 and the second blade 144.

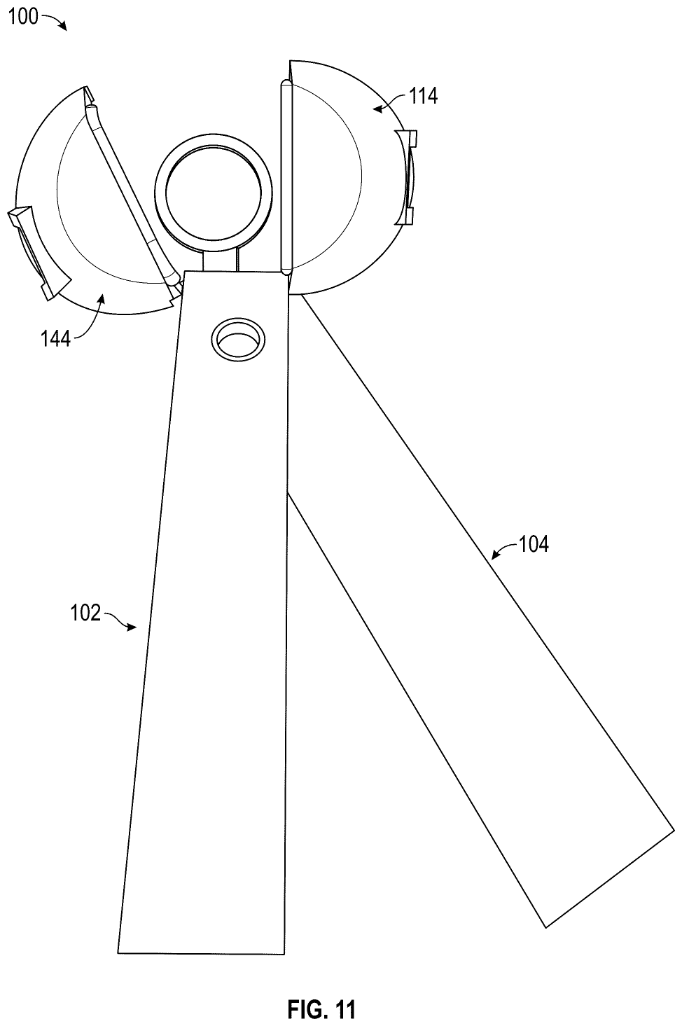

Referring now to FIGS. 1-11, one method of using the speculum may involve beginning with the speculum 100 in a closed state as seen in FIG. 10. The first blade distal end 118 and the second blade distal end 148 are placed in the entrance of the vaginal canal, and the speculum 100 can be guided the desired distance into the vaginal canal by the practitioner. While holding the first handle 108, the second handle 138 can be actuated away from the first handle 108 to cause the first blade 114 and the second blade 144 to separate so as to cause the speculum 100 to open, as seen in FIG. 11. When actuating the second handle 138 away from the first handle 108, the second blade 144 moves in a direction opposite of that in which the second handle 138 is moving. The first handle 108 and the second handle 138 are movable so as to transition the speculum 100 between the open state, as seen in FIG. 11, in which the first blade 114 and the second blade 144 are a first distance apart, and a closed state, as seen in FIG. 12, in which the first blade 114 and the second blade 144 are a second distance apart, where the first distance is greater than the second distance. To retain the speculum 100 in the open state, the practitioner may adjust the fastener 106 to apply tension between the first member 102 and the second member 104. The light 176 can be activated to improve visibility of the vaginal canal. The light 176 can be activated at any desired stage of the procedure or not at all. Once the procedure is done, the fastener 106 can be loosened to reduce the tension between the first member 102 and the second member 104 so as to cause the second handle 138 to actuate toward the first handle 108 to return the speculum 100 to the closed state. The speculum 100 can then be removed from the vaginal canal with minimal discomfort to the user. If the sleeve 200 is disposed on the speculum during the procedure, the sleeve 200 may then be disposed of.

Referring now to FIGS. 1-15, one method of using the speculum 100 may begin with the sleeve 200, or the sleeve members 302 and 304, disposed over the first blade 114 and the second blade 144 of the first member 102 and second member 104, respectively, and the speculum 100 in a closed state as seen in FIG. 10. The first blade distal end 118 and the second blade distal end 148 can be placed at the entrance of the vaginal canal. The speculum 100 can then optionally be pushed further into the vaginal canal at a desired distance. While holding the first handle 108, the second handle 138 can be actuated away from the first handle 108 (by pivoting around the fastener 106) resulting in the first blade 114 and the second blade 144 separating so as to transition the speculum 100 into an open state, as shown in FIG. 11. To retain the speculum 100 in an open state, the fastener 106 can be adjusted to apply tension between the first member 102 and the second member 104. The light 176 can then be activated to provide illumination of the vaginal canal. The light 176 can be activated at any desired stage of the procedure. Once the procedure is done, the fastener 106 can be loosened to reduce the tension between the first member 102 and the second member 104. The second handle 138 can then be actuated toward the first handle 108 to return the speculum 100 to the closed state. The speculum 100 can then be removed from the vaginal canal, and the sleeve 200 or sleeve members 302, 304 can be disposed of.

Certain embodiments of the devices and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the devices and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

What is claimed is:

1. A speculum comprising:
a first member having a first blade and a first handle, the first blade extending from a first blade proximal end to a first blade distal end;
a second member having a second blade and a second handle, the second blade extending from a second blade proximal end to a second blade distal end; and
a fastener configured to be disposed through the first handle and the second handle so as to secure the first member to the second member in a manner that allows the first blade and the second blade to open and close, the first handle and the second handle being movable so as to transition the speculum between an open state in which the first blade and the second blade are a first distance apart, and a closed state in which the first blade and the second blade are a second distance apart, wherein the first distance is greater than the second distance; and
a mount extending from a top surface of the first handle, the mount comprising a head portion and a neck portion extending between the top surface of the first handle and the head portion.

2. The speculum of claim 1, wherein the first blade is tapered from a first blade proximal end to a first blade distal end, and the second blade is tapered from a second blade proximal end to a second blade distal end.

3. The speculum of claim 2, wherein the tapering of the first blade is uniform and the tapering of the second blade is uniform.

4. The speculum of claim 1, wherein the first blade is curved and the second blade is curved.

5. The speculum of claim 1, wherein:
the first handle has a first handle distal end and the first blade a first blade inferior edge, and the first blade inferior edge is attached to a first corner of the first handle distal end; and
the second handle has a second handle distal end and the second blade has a second blade inferior edge, and the second blade inferior edge is attached to a second corner of the second handle distal end.

6. The speculum of claim 1, wherein the first blade has a first convex surface, the second blade has a second convex surface, and either the first convex surface or the second convex surface includes at least one marking configured to serve as a measurement indicator.

7. The speculum of claim 1, wherein the head portion is circular and defines an opening configured to receive a light.

8. The speculum of claim 7, wherein the opening is substantially centered within a tubular structure formed by edges of the first blade and the second blade when the speculum is in the closed state.

9. The speculum of claim 1, further comprising a light system disposed within the mount, wherein the light system comprises a light first portion and a light second portion, the light first portion being positioned on a mount distal side and the light second portion being positioned on a mount proximal side, wherein the light first portion houses a light and the light second portion houses a power source in electrical communication with the light.

10. The speculum of claim 1, wherein the fastener is configured to adjust tension between the first member and the second member.

11. The speculum of claim 1, wherein the first blade has a first length from the first blade proximal end to the first blade distal end, and the second blade has a second length from the second blade proximal end to the second blade distal end, the second length being longer than the first length.

12. The speculum of claim 1, wherein the first blade has a first convex surface, the second blade has a second convex surface, and the first convex surface and the second convex surface are smooth.

13. The speculum of claim 1, further comprising a sleeve covering at least a portion of the first blade and/or the second blade.

14. The speculum of claim 13, wherein the sleeve is silicone or latex.

15. The speculum of claim 1, wherein the fastener comprises a nut and bolt.

16. A method of examining a body cavity, the method comprising:
actuating first and second handles of a speculum so as to cause first and second blades of the speculum to move closer together, thereby causing the speculum to be in a closed state;
inserting a distal end of the first and second blades into the body cavity;
moving the first and second blades a desired amount into the body cavity;
allowing the first and second handles to move farther apart so as to cause the first and second blades to move farther apart in a horizontal direction relative to the body cavity, thereby causing the speculum to be in an open state; and
examining the body cavity.

17. The method of claim 16, further comprising illuminating the body cavity with a light mounted at the distal end of the speculum.

18. The method of claim 16, further comprising actuating the first and second handles so as to cause the first and second blades to move closer together, thereby causing the speculum to be in the closed state, and removing the first and second blades from the body cavity.

19. A speculum comprising:
first and second blades extending in a direction orthogonal to first and second handles, the handles being pivotable around aligned bores receiving a fastener, the fastener being configured to lock or unlock the first and second handles in position relative to one another;
wherein the first and second blades are moved closer to one another as the first and second handles are pivoted to a greater extent; and
wherein the first and second blades are curved so as to define a tubular structure when the speculum is in a closed state.

20. The speculum of claim 19, further comprising a light mounted to the first handle at a distal end of the tubular structure.
